(19) United States
(12) Patent Application Publication
(10) Pub. No.: US 2011/0038882 A1
(43) Pub. Date: Feb. 17, 2011

(54) Methods for Treating Allergic Disease

(75) Inventors: Bor-Luen Chiang, Taipei City (TW); Chun-Jung Chang, Taipei City (TW); Yu-Li Lin, Taipei City (TW); Kuan-Hua Chu, Taipei-City (TW); Hong-Nong Chou, Taipei City (TW); Jiunn-Ming Jeng, Taipei City (TW)

Correspondence Address: FRENKEL & ASSOCIATES, 3975 UNIVERSITY DR., STE. 330, FAIRFAX, VA, 22030, US

(73) Assignee: National Taiwan University, Taipei (TW)

(21) Appl. No.: 12/855,132

(22) Filed: Aug. 12, 2010

Related U.S. Application Data
(60) Provisional application No. 61/234,366, filed on Aug. 17, 2009.

(51) Int. Cl. A61K 38/16; A61P 37/08; A61P 11/06; A61P 11/02; A61P 17/06; A61P 17/00; A61P 29/00; A61P 1/00; A61P 11/08; A61P 37/02

(52) U.S. Cl. 424/184.1

(57) ABSTRACT

A method for treating or alleviating allergic disease in a mammal in need thereof having administering to the mammal a therapeutically effective amount of a pharmaceutical composition having phycocyanin is provided. A method for modulating balance between Th1 and Th2 immune response in a mammal in need thereof, having administering to the mammal an effective amount of phycocyanin, wherein immune response of the mammal is skewed toward the Th1 immune response is also provided. Phycocyanin from Bangia atropupurea (Ba-PC) is identified to regulate mammalian immunological response indicating that Ba-PC can direct skewed immune response toward Th1 response through modulating DC function, increase proliferative activity of antigen-specific T cells and alleviate airway inflammation, confirming that phycobiliproteins are effective in treating allergic disease.

### CROSS-REFERENCE TO RELATED APPLICATION

This application claims the benefit of U.S. Provisional Application No. 61/234,366, filed on Aug. 17, 2009, the contents of which are hereby incorporated by reference in its entirety.

### BACKGROUND OF THE INVENTION

1. Field of the Invention

The present invention relates to an application of phycobiliprotein for modulating immune response, more particularly to the application of phycocyanin from Bangia atropupurea (Ba-PC) in allergic disease treatment.

2. Description of the Prior Arts

Phycobiliproteins are water-soluble proteins present in cyanobacteria and certain algae (rhodophytes, cryptomonads, glaucocystophytes). Phycobiliproteins are formed as a complex between proteins and covalently bound phycobilins (such as phycocyanobilin, phycoerythrobilin, phycourobilin and the like) that act as chromophores. Each phycobiliprotein has a specific absorption and fluorescence emission maximum at visible light wavelengths. In this way, the cells take advantage of available wavelengths of light (ranging from 500-650 nm), which are inaccessible to chlorophyll, and utilize their energy for photosynthesis.

Phycocyanin (PC) consists of three α-subunits (18,800 Da) and three β-subunits (20,100 Da) in a form of a trimeric aggregation (αβ)₃. Phycocyanin is usually obtained from plants such as algae and is safe when used in food, drink and cosmetics as a coloring agent. Easily grown, blue-green algae such as Spirulina and Microcystis are major raw materials for phycocyanin Another abundant natural marine resource of phycocyanin is red algae, such as Porphyra and Ceramium.

Pure phycocyanin has been applied in many fields due to being known for photochemical effects of the molecule when irradiated with a suitable wavelength of light. For example, phycocyanin can be used in fluorescent labeling of antibodies applied as diagnostic agents in immunological, clinical, cell biological and biochemical research. Since methods of algae cultivation and preparation of phycocyanin from blue-green algae have been well established, phycocyanin is considered a potential candidate of therapeutic agents compared to other synthetic pharmaceutical agents.

Allergic diseases caused by immune response occurring to normally harmless environmental substances known as allergens, wherein the reactions are acquired, predictable, and rapid Taking asthma, an well-known allergic disease, as an example, the pathophysiology of asthma is characterized by eosinopilic inflammation of the airways, bronchospasm, and hyperreactivity to nonspecific inhaled stimuli. Asthma has been characterized by an imbalance between Th1 and Th2 lymphocytes and by a predominant Th2-type immune response. Th2-type cytokines, such as interleukin-4 (IL-4), IL-5 and IL-10, may lead to eosinophilic and mast cell chemotaxis and activation, as well as B-cell production of IgE. Some studies have demonstrated that the induction of Th2-type cytokines and proliferation of committed Th2 lymphocytes can be prevented by Th1-type cytokines, notably interferon-γ (IFN-γ) and IL-12.

However, no literature or publications have explored and disclosed the effects of phycobiliproteins including phycocyanin on modulation of immune response, especially on promotion of Th1 cytokine response and inhibition of Th2 response. Needless to say, little is known about an efficacy of phycocyanin in treatment of allergic disease. To meet urgent needs for developing novel therapeutic compositions or methods for treating allergic disease, efficacy of phycobiliproteins, especially phycocyanin from Bangia atropupurea (Ba-PC), in treatment of allergic disease is explored.

### SUMMARY OF THE INVENTION

Accordingly, applicants endeavor to develop a method for treating allergic disease by using phycobiliproteins, particularly by using phycocyanin from Bangia atropupurea for treating allergic disease. The applicants establish a platform for screening compounds that acquire ability to enhance immunity and could be promising target for treatment of allergic diseases. Bangia atropupurea (Ba-PC) herein is identified to regulate mammalian immunological response through both in vivo and in vitro experiments confirming that phycobiliproteins, particularly phycocyanin, are effective in treating allergic disease.

Therefore, in one aspect, the present invention provides a method for treating or alleviating allergic disease comprising administering to a mammal suffering from allergic disease a therapeutically effective amount of a pharmaceutical composition comprising phycocyanin.

In another aspect, the applicants apply a method for evaluating the immune modulatory function in vitro through BMDC culture system to monitor the efficacy of novel molecules in immune regulation in a mammal in need thereof.

Therefore, the present invention also provides a method for modulating balance between Th1 and Th2 immune response in a mammal in need thereof comprising:

administering to the mammal an effective amount of phycocyanin, whereby immune response of the mammal is skewed toward the Th1 immune response.

In another aspect, the present invention also provides a pharmaceutical composition for treating or alleviating allergic disease comprising a therapeutically effective amount of phycocyanin and a pharmaceutically acceptable carrier or excipient therefor.

In yet another aspect, the present invention also provides a supplementary food composition for treating, preventing or alleviating allergic disease comprising an appropriate amount of phycocyanin.

Since phycocyanin can be prepared at low cost, are safe for drinking and eating, and are confirmed to be effective in treating allergic disease, the method and the composition for treating or alleviating allergic disease according to the present invention have great advantages over other conventional methods or therapeutic agents for treating or alleviating allergic disease.

Other objectives, advantages and novel features of the invention will become more apparent from the following detailed description when taken in conjunction with the accompanying drawings.

### BRIEF DESCRIPTION OF THE DRAWINGS

FIG. 1 illustrates absorption spectrum of phycocyanin isolated from Bangia atropupurea (Ba-PC);

FIG. 2 illustrates fluorescence spectrum of Ba-PC;

FIG. 3 illustrates results of analysis by native-PAGE (6%) of Ba-PC (panel A); and results of analysis by SDS-PAGE of Ba-PC (panel B), wherein left lanes shows molecular weight markers of 200, 116.25, 97.4, 66.2, 45.0, 31.0, 21.5, 14.4 and 6.5 kDa, and right lane shows α and β subunits of 17.06 and 22.69 kDa respectively;

FIG. 4 illustrates IL-12 p70 production of dendritic cells (DCs) treated with or without Ba-PC crude extract (FIG. 4A), purified Ba-PC (FIG. 4B) and endotoxin-free Ba-PC crude extract (FIG. 4C);

FIG. 5 illustrates expression level of surface markers as indicated of DCs incubated with Ba-PC (75 μg/ml), wherein LPS represent DCs cultured with LPS (0.1 mg/ml), which served as a positive control;

FIGS. 6A and 6B illustrate IL-12 p40 production in different groups of DCs treated with indicated molecules (LPS, PS-G or Ba-PC), the DCs cultured with culture medium served as negative control (untreated group) and DCs cultured with LPS or PS-G served as positive controls;

FIG. 7 illustrates IL-10 production in different groups of DCs treated with indicated molecules (LPS, PS-G or Ba-PC);

FIG. 8 illustrates capacity of endocytosis of DCs treated with Ba-PC by analyzing uptake of fluorescein isothiocyanate (FITC) labeled dextran by DCs (*p<0.05), LPS-treated DCs were used as positive control and untreated DCs were used as negative control;

FIG. 9 illustrates effects of Ba-PC-treated DCs on stimulating CD4⁺ T cell proliferation in mix lymphocyte reaction (MLR), compared to untreated group (mock), Ba-PC-treated DCs are able to induce higher levels of proliferation of CD4⁺ T cells derived from naive C57BL/6 mice (*p<0.05), wherein LPS-treated DCs were used as positive control;

FIGS. 10A to 10C illustrate IFN-γ and IL-4 production of CD4⁺ T cells stimulated with Ba-PC-treated DCs after 48 hours (FIG. 10A), 72 hours (FIG. 10B) and 96 hours (FIG. 10C), herein, LPS-treated DC served as positive control and untreated DC act as negative control after initial allogenic mixed lymphocyte reactions as described in Example 4;

FIGS. 11A and 11B illustrate serum ovalbumin (OVA)-specific antibody levels (IgG1, IgG2a in FIG. 11A, and IgE in FIG. 11B) in different experimental groups as described in Example 5 (*: p<0.05, **: p<0.001), wherein EU is represented arbitrarily in ELISA units of indicated antibody;

FIG. 12 illustrates inflammation in airway of the experimental mice as described in Example 5, mice were immunized with OVA alone or OVA in combination with Ba-PC or PS-G through i.p. injection at day 0, day 5, day 10, day 15, day 20 and day 25 following treatment with OVA through air way inhalation at 24 h, 48 h, 72 h and 96 h after last immunization, mice were sacrificed and lung tissues were collected to perform paraffin embedding following H & E staining, mice treated with OVA in combination with PS-G (PS-G combinational-treated group) served as a positive control, compared to Ba-PC and untreated group, the upper panels shown that the lung tissues derived from mice immunized with OVA alone reveal significant bronchia epithelium thickness, leukocytes infiltration and bronchia inflammation. Lower panels of FIG. 12 show enlarged figures of corresponding upper panels of FIG. 12;

FIGS. 13A and 13B illustrate cell compositions in bronchoalveolar lavage fluid (BALF) obtained from OVA-immunized mice with different treatments as described in Example 6, FIG. 13A illustrates cell number of eosinophils in BALF, FIG. 13B illustrates percentage of eosinophil in BALF (***: p<0.0001);

FIG. 14 illustrates airway hyperresponsiveness in OVA-immunized mice treated with different treatments as described in Example 7 (**: p<0.001);

FIGS. 15A to 15E illustrate levels of Th2 cytokine, such as IL-4 (A), IL-10 (B), IL-5 (C), eotaxin (D) and IL-13 (E), in brochoalveolar lavage fluid from OVA-immunized mice with different treatments as described in Example 8 (*: p<0.05, **: p<0.001, ***: p<0.0001);

FIGS. 16A to 16E illustrate levels of Th2 cytokine, such as IL-4 (A), IL-5 (B), IL-10 (C), IL-13 (D) and IFN-γ (E), by splenocytes obtained from each group of experiments as described in Example 9 (*: p<0.05, **: p<0.001, ***: p<0.0001); and

FIG. 17 illustrates proliferative activity of antigen-specific T cells obtained from mice treated with OVA in combination with Ba-PC or PS-G as described in Example 10 (*: P<0.05).

### DETAILED DESCRIPTION OF THE PREFERRED EMBODIMENTS

Phycobiliproteins include phycocyanin obtained from Bangia atropupurea (Ba-PC), and are identified herein to regulate mammalian immunological response through both in vivo and in vitro experiments. Experiment of in vitro analysis is performed by the following method: fresh bone marrow cells were collected from femur and tibia of female BALB/c mice and cultured in RPMI 1640 medium containing 500 U/ml GM-CSF and 1000 U/ml IL-4 for 6 days; then phycocyanin were supplemented into dendritic cells (DCs) on day 6; on day 7 or day 8, supernatant was collected for detecting IL-12p40, which was known to play an important role in Th1 cell differentiation, here the applicants established an in vitro system for the screening of potential active compounds for immune enhancing ability and possibly applying the same to the treatment of allergic diseases and proved that phycobiliproteins are effective in treating allergic disease, for example, but not be limited to, allergic asthma, allergic rhinitis and allergic pneumonia.

Regarding methods for modulating imbalance between Th1 and Th2 immune response to alleviate allergic disease, a lineage of immune cells drawing attention of researchers in the field are dendritic cells (DCs). DCs, as professional antigen-presenting cells, are most efficient activators of resting T cells and have a unique capacity to activate naïve T cells. Due to ability of DCs to modulate development of T cells and promote development of Th1 or Th2 immune response toward specific antigens, DCs have been utilized as an effective modulator for alleviating airway inflammation through pulsing with different antigens.

As for phycocyanin sources, red algae were subject to extraction to obtain desired phycocyanin in the present invention. As known, most red algae contain a high gel content, making extraction of phycocyanin very difficult, especially for dried algae. The applicants obtained phycocyanin from Bangia atropurpurea by a process for preparing phycocyanin from filamentous phase (Conchocelis) of Bangia atropurpurea. Briefly, the applicants obtained clean non-polluted algal biomass from filamentous tissue culture that were developed from carpospore germination under a light- and temperature-controlled growth system. Biliproteins of Bangia atropurpurea are isolated from the algal biomass by a cost-effective extraction and a series of chromatographic separations.

As used herein, the term “allergic disease” refers to allergic reaction against an allergen derived from, for example, but not limited to self-antigen, ragweed, birch pollen, peanut, house dust mite, animal dander, mold and tropomyosin.

According to the present invention, the allergic disease includes, but not limited to: asthma, allergic rhinitis, eczema, psoriasis, atopic dermatitis, rheumatoid arthritis, inflammatory bowel disease, Crohn's disease, ulcerative colitis, chronic obstructive pulmonary disease, conjunctivitis, nasal congestion and urticaria.

As used herein, the term “allergic airway disease” means allergic disease of the airways. For example, allergic airway disease comprises, but is not limited to: asthma, allergic rhinitis and allergic pneumonia.

According to the present invention, the phycocyanin is obtained from alga selected from the strains belonging to the species of Bangia atropurpurea, Porphyra angusta, Porphyra dentata, thereof. Preferably, the phycocyanin is obtained from Bangia atropupurea (Ba-PC).

In one aspect, the present invention provides a method for treating or alleviating allergic disease in a mammal in need thereof, comprising:

administering to a mammal suffering from allergic disease a therapeutically effective amount of a pharmaceutical composition comprising phycocyanin.

In a preferred example of the present invention, the pharmaceutical composition is administered orally, inhalationally or intranasally to the mammal.

In another preferred example of the present invention, the pharmaceutical composition is administered intravenously, subcutaneously, or intramuscularly to the mammal.

According to the method of the present invention, the therapeutically effective amount is between 0.1 mg per kg per day and 50 mg per kg per day.

In another aspect, the present invention provides a method for modulating balance between Th1 and Th2 immune response in a mammal in need thereof, comprising:

administering an effective amount of phycocyanin to the mammal, wherein immune response of the mammal is skewed toward Th1 immune response.

According to the present invention, as used herein the term “immune response being skewed toward the Th1 immune response” refers to production of Th2 type cytokines, such as IL-4, IL-10, IL-5, eotaxin and IL-13, which tend to be decreased, and production of Th1 type cytokines such as IFN-γ, which tend to be increased in presence of phycocyanin.

According to the method of the present invention, mammals with immune response skewed toward the Th2 immune response suffer from allergic disease. According to the method of the present invention, the effective amount is between 0.1 mg per kg per day and 50 mg per kg per day.

In yet another aspect, the present invention provides a pharmaceutical composition for treating or alleviating allergic disease comprising a therapeutically effective amount of phycocyanin and a pharmaceutically acceptable carrier or excipient therefor.

According to the method of the present invention, the therapeutically effective amount is between 0.1 mg per kg per day and 50 mg per kg per day.

In a preferred example of the present invention, the pharmaceutical composition is formulated for administration by oral, topical, parenteral, intramuscular, intranasal, subcutaneous or intravenous routes. More particularly, the pharmaceutical composition is a powder, tablet, pill, capsule, cachet, suppository, plurality of dispersible granules, suspension, microemulsion or the like.

The pharmaceutical composition of the present invention may be formulated into any suitable dosage form, such as tablet, capsule, pill, lozenge, granule, powder, pellet, liquid, emulsion, suspension, elixir or the like. The pharmaceutical composition may be packaged with a propellant in a pressurized aerosol container within an inhaler, or a nasal sprayer.

According to the present invention, the pharmaceutically acceptable carrier or excipient are well known in the art and include phosphate buffered saline solutions, water, emulsions, such as oil/water emulsions, various types of wetting agents, sterile solutions etc. The excipient may be any pharmaceutical excipient that would function as carrier material, bulking agent, binder, lubricant, buffer, surfactant, diluent, disintegrant, glidant, colouring agent or the like.

In yet another aspect, the present invention provides a supplementary food composition for preventing or alleviating allergic disease comprising an appropriate amount of phycocyanin.

In the present invention “an effective amount” of the phycocyanin will vary with individual patients and severity of disease, however, generally the effective amount will be at least about 0.1 mg per kg. Preferably, the appropriate amount of phycocyanin is between 0.1 mg per kg and 50 mg per kg.

The present invention was further illustrated by the following examples, but it should be understood that the examples and embodiments described herein are for illustrative purposes only, and should not be construed as limiting to the embodiments set forth herein.

### Examples

### General Experimental Materials and Methods

1. Animal

Female BALB/c mice aged 6 to 8 weeks and weighing around 20 g were maintained in the Animal Center of the College of Medicine of National Taiwan University (Taipei, Taiwan). The animal study protocol was approved by the Animal Research Committee of College of Medicine, National Taiwan University.

2. Preparation of Ba-PC

Mature Bangia atropurpurea thalli were collected from sea and washed with sterilized seawater. After a short time of air-drying, they were placed into culture medium (SWM-III medium). After a few hours, carpospores would be released from the Bangia atropurpurea thalli. The released spores in medium were then removed from their thalli and placed in a growth chamber wherein the temperature, illuminance and light/dark ratio were 25° C., 500-1000 lux and 14:10 respectively.

After the spores germinated to branched filaments (Conchocelis), the filaments were transferred to SWM-III medium-containing flasks, and cultivated in the above condition until they formed colonies as filamentous clusters. The filamentous clusters were propagated by fragmentation through blending of a sterilized grinder. The growth of small cutting segments of the filament were enhanced by transferring to a new SWM-III medium in a larger volume, such as a fiberglass tank or concrete tank through a series of scaling-up process.

For each step of scaling-up, the filamentous colonies were cut again for further growth in a larger volume until the required amount was acquired. Note that when the filamentous colonies were cultivated in a large flask or tank, fresh air (300 ml air/min) must be supplied to the tank. This was for the supply of CO₂ for growth and also for agitation purposes. The filaments were then collected and filtered by a net of 100-400 mesh. The culture medium if not contaminated by other algae could be recovered and reused with enriched media ingredients. A higher density of culture or a deep culture vessel would need a high intensity top illumination such as 5000-10,000 lux on the culture surface or at least 500 lux at the bottom of culture vessels. An outdoor culture system would need to maintain water temperature below 30° C. by shading direct sun or by underground seawater cooling at noontime. A fast change of salinity of the culture medium, such as a dilution by quantities of rainwater or fresh water, should be avoided.

The collected and filtered Bangia atropurpurea filaments were then fast dried in a vacuum or by lyophilization, and then ground into powder form. The dried algal powder was added to a solution of 10 mM phosphate pH 6.8 or water (10 times in volume) and stirred vigorously to obtain a aqueous extract of proteins. Debris was removed by centrifugation to obtain a clear-red pigment solution, which was used as crude extract in the following examples. Repeated extractions could be performed to have a thorough extraction. Supernatants from subsequent extractions were combined and (NH₄)₂SO₄ crystals were added piece by piece with stirring until a 10% saturated solution was reached. After centrifugation to remove Chlorophyll-bound proteins, the supernatant was added with (NH₄)₂SO₄ crystals piece by piece with stirring until 30% saturated solution was obtained. The extract was centrifuged again to remove major phycoerythrin protein in precipitate. The supernatant was added with (NH₄)₂SO₄ crystals piece by piece with stirring again until a 50% saturated solution was obtained. Crude phycocyanin combined with some phycoerythrin was then obtained in precipitate by centrifugation.

Crude phycocyanin precipitate was resuspended using a trace amount of 10 mM phosphate solution and dialyzed against the mentioned phosphate solution by a dialysis tube and then subjected for a gel filtration chromatographic separation. The gel filtration chromatography used a Sephadex G200 column and the mentioned phosphate solution as eluent, eluted solution was collected into fractions according to peaks observed by UV 280 nm absorption using a photodiode array (PDA) detector. Fractions having a ratio of absorbance at 618 and 280 nm (A₆₁₈/A₂₈₀) larger than 2.5 were combined as crude phycocyanin. Those fractions having a ratio of A₅₆₅/A₂₈₀ larger than 1.4 were regarded as crude phycoerythrin. Through repeated Sephadex G200 gel filtration chromatography, the absorption ratio A₅₆₅/A₂₈₀ of phycoerythrin fractions increased until reaching purity A₅₆₅/A₂₈₀>5.1, while phycocyanin was further purified. The combined crude phycocyanin fractions from gel filtration chromatography were precipitated in a 50% saturated solution of (NH₄)₂SO₄. Then the dialyzed crude phycocyanin solution was subjected for the further hydroxyapatite chromatography purification using a self-packed Macro-Prep Ceramic Hydroxyapatite Type I gel (Bio-Rad) (i.d. 2.5×20 cm) column. Phycocyanin was eluted by a linear gradient elution of 10 mM to 200 mM phosphate in 0.1 M NaCl solution. Eluted fractions were monitored by UV-Visable spectrophotometer. Purified phycocyanin was combined from the fractions that having ratios A₆₁₈/A₂₈₀>4 and precipitated by adding (NH₄)₂SO₄ crystals to dissolve until a solution of 60% saturation was reached.

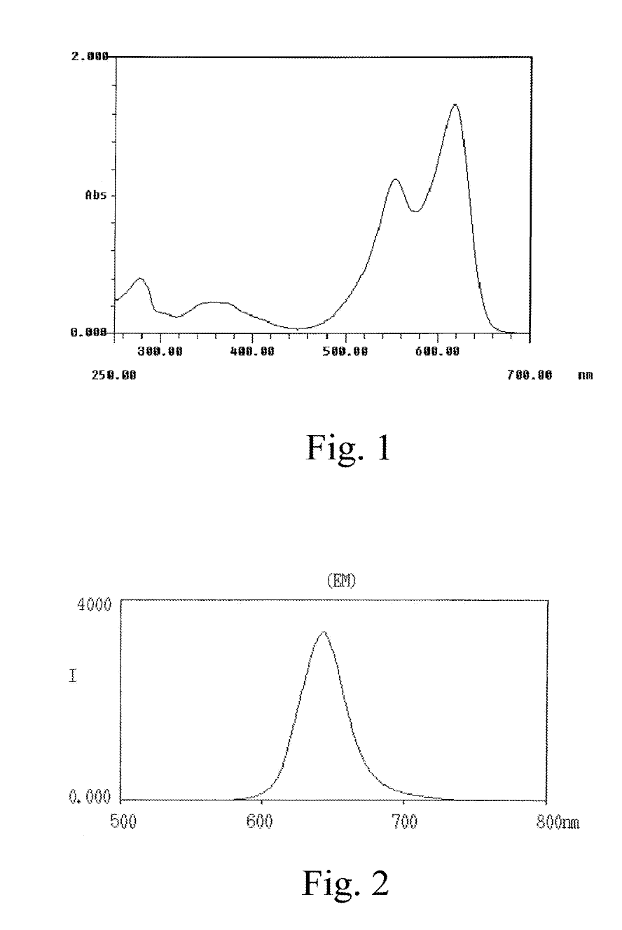

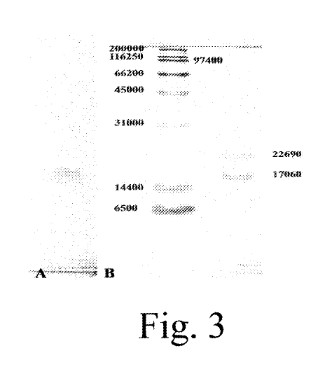

Phycocyanin from Bangia atropurpurea (Ba-PC) belonged to type I R-PC, which had maximal absorption λₘₐₓ at 618 nm and then 553 nm, and emitted fluorescence wavelengths of 640 nm (FIG. 1 and FIG. 2). The results of native- and SDS-PAGE showed Ba-PC was a conjugate of (αβ)₃, 3× α (MW 17.06 kDa) and 3×β (MW 26.69 kDa) subunits (FIG. 3). Ba-PC isolated through above methodologies and characterized here was subject to the following assay of airway hyperresponsiveness in mice and dendritic cell bioassay.

3. Isolation and Modulation of Mouse Dendritic Cells from Bone Marrow Cultures

Bone marrow derived dendritic cells (BMDCs) were prepared as described previously (Richards D F. et al., Eur J. Immunol., 2000, 30: 2344-54). Briefly, bone marrow cells from femurs and tibias were depleted of red cells by using an ACK lysis buffer and cultured, 1.5×10⁶ cells were placed in 24-well plates in 1 ml of medium that was supplemented with recombinant murine GM-CSF (mouse granulocyte macrophage-colony-stimulating factor) (500 unit/ml) and IL-4 (1000 unit/ml) (Pepro Tech Inc., Rocky Hill, N.J.). The culture medium was RPMI-1640 medium supplemented with 5% heat-inactivated foetal calf serum, 4 mM L-glutamine, 25 mM HEPES (pH 7.2), 50 μM 2-mercaptoethanol, 100 unit/ml penicillin, 100 μg/ml streptomycin and 0.25 μg/ml amphotericin. Every other day, the medium was removed and fresh medium was added. Mouse BMDCs were harvested on day 6 for further experiments. On day 6 of culture, non-adhered cells (BMDCs) were collected and treated with or without LPS (0.1 μg/ml) or Ba-PC (75 μg/ml). On day 8, cultured supernatant and LPS, Ba-PC-treated and untreated BMDCs were harvested and prepared for analyzed.

4. Flow Cytometry

Cells for analysis were washed with cold buffer PBS (phosphate-buffered saline) containing 2% fetal calf serum (FCS) and 0.1% sodium azide and then incubated in cold buffer. Subsequently, cells were stained with rat, anti-mouse, monoclonal antibodies to isotype controls, IA^{d} (MHC class II), CD80 (B7-1), CD86 (B7-2), CD40, CD205 or CD11c (eBioscience, San Diego, Calif.) for 30 mins on ice. Stained cells were then washed twice and resuspended in cold buffer and a FACS Calibur (Becton Dickson) was used for analytical flow cytometry and data were processed with CellQuestPro (Becton Dickson) software.

5. Determination of Cytokine Expression

For biological function analysis of BMDCs, expressional levels of IL-12 p40, IL-12 p70, IL-4, IL-10 and IFN-γ were assayed by ELISA according to the manufacturer's recommended instructions (R&D, Minneapolis, Minn., USA).

6. Analysis of DC Endocytosis

To evaluate maturation status of DCs after Ba-PC treatment, the capability of endocytosis of DCs were determined by fluorescein isothiocyanate (FITC) labeled dextran uptake. Ba-PC treated mouse bone marrow derived DCs were harvested and washed twice and resuspended in 1 ml RPMI 1640 supplemented with 10% FCS, 2 mM L-glutamine, 100 U/ml penicillin, 100 U/ml streptomycin, and 25 mM HEPES. The cells were then incubated with FITC-labeled dextran (0.2 mg/ml) at 4° C. or 37° C. for 1 hr. Finally, the cells were washed three-times with cold buffer and analyzed with a FACSCalibur (Becton Dickson) as described above.

7. Allogenic Mixed Lymphocyte Reactions and Cytokine Production Analysis

To examine stimulating ability of Ba-PC-treated DCs, mixed lymphocytes reaction (MLR) and cytokine-secreting levels were determined. CD4⁺ T cells were purified from spleens of C57BL/6 mice by magnetic sorting with L3T4 (anti-CD4) bound to magnetic beads and MiniMACS columns (Miltenyi Biotec, Bergisch Gladbach, Germany) according to the manufacturer's instructions. Positively selected cells containing 95 to 99% CD4⁺ cells were collected for cytokine production and proliferation assay.

For the proliferation assay, Ba-PC or LPS treated BMDCs were irradiated at 3000 rads and used as APCs. Freshly isolated C57BL/6 CD4⁺ T cells (3×10⁵ cells/ml) were co-cultured with Ba-PC or LPS treated BMDCs at an indicated DC/T cell ratio. Cells were cultured in a total volume of 200 μl in 96-well, round-bottom tissue culture plates for 3 days. Cultures were then pulsed with 1 μCi [³H] thymidine for another 17 hours of culture and the [³H] thymidine deoxyribose incorporation was measured by scintillation counter. For cytokine analysis, Ba-PC treated BMDCs were cultured under the same conditions as used in the proliferation assay. After 48, 72 and 96 hours, supernatants were collected and cytokine production was analyzed by ELISA.

8. Intracellular Cytokine Staining

Intracellular cytokine were detected by flow cytometry using the method of Andersson et al. (Andersson U. et al., Eur. J. Immunol., 1990, 20: 1591-6) with modifications. Briefly, 3×10⁵ CD4⁺ T cells and DC were incubated at an indicated DC/T cell ratio at 37° C. for 2 days. Monensin was added for the final 6 hours. Then, 10⁶ cells were harvested and stained with phycoerythrin-labeled monoclonal antibodies to CD4 (BD PharMingen, San Diego, Calif.). Subsequently, cells were washed, fixed and permeabilized using eBioscience IC Fixation Buffer/eBioscience Permeabilization Buffer (eBioscience, San Diego, Calif.) and stained with fluorescein isothiocyanate (FITC) labeled monoclonal antibodies to IFN-γ or IL-4 (BD Biosciences Pharmingen). Staining with isotype control antibodies (BD PharMingen, San Diego, Calif.) was performed in all experiments. 10⁴ of cells were analyzed by flow cytometry. FACS analyses were shown after gating on the CD4⁺ lymphocyte population.

9. Induction of Allergic Airway Inflammation with OVA and Analysis of Airway Hyperresponsiveness (AHR)

Female 6-to-8-week-old BALB/c mice were sensitized by an intraperitoneal injection of 50 μg of OVA (Sigma, St Louis, Mo., USA) mixed with 50 μg or 100 μg of Ba-PC in a total volume of 200 μl PBS (hereafter referred to as “mice treated with OVA in combination with Ba-PC”) on day 0, days 5, 10, 15, 20 and 25. PS-G (polysaccharide from G lucidum) was kindly provided by Professor Shiuh-Sheng Lee of Department Of Biochemistry of National Yang-Ming University and used as a positive control (in vitro study for a dose of 10 μg/ml; in vivo study for a dose of 1 mg/ mice). Applicant's previous study (Lin, Y L. et al., Journal of Leukocyte Biology vol. 78: 533-43, 2005) demonstrated that PS-G can promote the activation and maturation of immature DC and PS-G may yield potential in regulating immune responses. Therefore, PS-G was used as a positive control in the study.

Mice of disease group (denoted as “OVA” in figures) were sensitized with OVA alone. Mice of naïve group (denoted as “untreated” in figures) were administered with PBS. For induction of allergic airway inflammation by OVA challenge, all mice were intranasally administered with OVA (50 μg in a total volume of 30 μl PBS) on days 35, 36, 37, 38. Mice of naïve groups were administered with PBS as negative control. Blood was collected by retro-orbital puncture at various time points during immunization period to analyze titer of IgE. Twenty-four hours after last OVA challenge, airway hyperresponsiveness (AHR) was assessed, then sera, bronchoalveolar lavage fluid (BAL fluid) and spleen were collected.

Partial lungs were cut and fixed with 10% neutralized buffered formalin. Sections (5 μm thick) of lung were prepared and subjected to Hematoxylin and Eosin (H&E) staining and examined by light microscopy. Single cell suspensions of splenocytes were made by pressing spleen tissue through a 40-μm mesh sieve and washing twice with Hank's balance buffer. Cells obtained were used for in vitro stimulation with OVA and the cytokine production was analyzed by enzyme-linked immunosorbent assay (ELISA).

10. Detection and Determination of OVA-Specific Antibody

Sera anti-OVA IgE, IgG1 and IgG2a antibody titers were determined by ELISA. Briefly, 96-well microtiter plates were coated with 1 μg per well OVA in NaHCO₃ buffer (pH 9.6) after incubation at 4° C. overnight, plates were washed and blocked with 3% bovine serum albumin in PBS for 2 hours at room temperature. Serum samples were collected, diluted and added to each well and incubated at 4° C. overnight. Plates were then washed.

Biotin-conjugated anti-mouse IgG1 (1:5000; BD PharMingen, San Diego, Calif.), anti-mouse IgE (1:1000; BD PharMingen, San Diego, Calif.) or IgG2a (1:1000; BD PharMingen, San Diego, Calif.) were diluted in 3% bovine serum albumin-PBS buffer and added, followed by incubating for 45 mins at room temperature. Streptavidin-conjugated horseradish peroxidase (1:5000; Pierce Biotechnology, Rockford, Ill., USA) was added and incubated for an additional 30mins at room temperature. Finally, the reaction was developed using peroxidase substrate, 2,2′-azino-bis(3-ethylbenzothiazoline-6-sulfonic acid) diammonium salt TMB (Clinical Science Products, Mass., USA), followed by addition of H₂SO₄ stop solution and determination of absorbance under a wavelength of 450 nm in a microplate reader (Molecular Devices, USA). Levels of antibody were compared to standard serum, calculated and expressed in arbitrarily ELISA units (EU):

EU=(Aₛₐₘₚₗₑ−A_{blank})/(Aₚₒₛᵢₜᵢᵥₑ−A_{blank});

Aₛₐₘₚₗₑ represents absorbance of sample;

A_{blank} represents absorbance of 3% bovine serum albumin-PBS; and

Aₚₒₛᵢₜᵢᵥₑ represents absorbance of standard serum derived from mice with high severity score of disease.

11. Determination of Airway Function

Airway function was measured by changes in RL in response to increasing doses of aerosolized MCh (3.125 to 25 mg/ml; Sigma, St Louis, Mo., USA) in anesthetized mice using a modification of the techniques described by Glaab et al. (Glaab, T. et al., J. Appl. Physiol., 2004, 97: 1104-1111.) Mice were anesthetized with ketamine hydrochloride (90 mg/kg; Phizer), tracheostomized and mechanically ventilated at a rate of 150 breaths per min, a tidal volume of 0.3 ml and a positive end-expiratory pressure of 3 to 4 cm H₂O with a computer controlled small animal ventilator (Harvard Rodent Ventilator, model 683; Harvard Bio-Science, Southnatick, Mass., USA). PE-50 tubing was inserted into the esophagus to the thorax and coupled with a pressure transducer (LDS GOULD, Valley View, Ohio, USA). Flow was measured by electronic differentiation of volume signal. Pressure, flow and volume changes were recorded. Pulmonary resistance was calculated by a software program (Model PNM-PCT100W, LDS PONEMAH Physiology Platform; LDS GOULD). MCh aerosol was generated with an in-line nebulizer and administrated directly through the ventilator. The resistance of the orotracheal tube (0.45 cm H₂O s/ml) was subtracted from all airway resistance measurements. Data were expressed as RL in the ratio of RL after PBS nebulization of three independent experiments.

12. Preparation and Analysis of Cellular Composition and Cytokine Level of BAL Fluid

Bronchoalveolar lavage fluid (BAL fluid) was removed by cannulation of the trachea of each mouse and washing airways with 1 ml Hank's balanced salt solution. The BAL fluid was centrifuged at 1500 r.p.m. for 10 mins at 4° C. and supernatant was stored at −20° C. to determine production of IL-4, IL-10, IL-5, IL-13, eotaxin and IFN-γ by ELISA. Pellets of cells were resuspended with secondary collected BAL fluid, which was collected from trachea washing in Hank's buffered salt solution supplemented with 2% of FCS. Appropriate cells of BAL fluid (about 2×10⁴) were cytospined and stained with Liu's stain. Based on morphology, a minimum of 200 cells were counted and classified as macrophages, lymphocytes, neutrophils or eosinophils to analyze inflammatory cell population in BAL fluid.

13. Statistical Analysis

One-way ANOVA comparison test was used to evaluate statistical significance of differences between experimental groups. P-values less than 0.05 were considered to be significant. All results were expressed as means±SE.

### Example 1

### Preliminary Analysis of Cytokine Production of Ba-PC Crude Extract-Pulsed Bone Marrow Derived Dendritic Cells

Ba-PC crude extract was obtained as described in “2. Preparation of Ba-PC”. Ba-PC crude extract-treated and untreated bone marrow derived dendritic cells (BMDCs) were prepared and cultured as described in “3. Isolation and modulation of mouse dendritic cells from bone marrow cultures”. BMDCs were treated with or without 18.8 μg/ml of Ba-PC crude extract for 48 hours. For comparison, BMDCs were also treated with 12.5 μg/ml of Ba-PC crude extract free of endotoxin for 48 hours. The Ba-PC crude extract without endotoxin (denoted as endotoxin-free Ba-PC) was prepared by passing the Ba-PC crude extract through an endotoxin removing column [Detoxi-Gel Endotoxin Removing Gel (PIERCE)] per the manufacture's manual. The cultured conditional medium obtained at 48 hrs after initial culturing was collected and analyzed by ELISA.

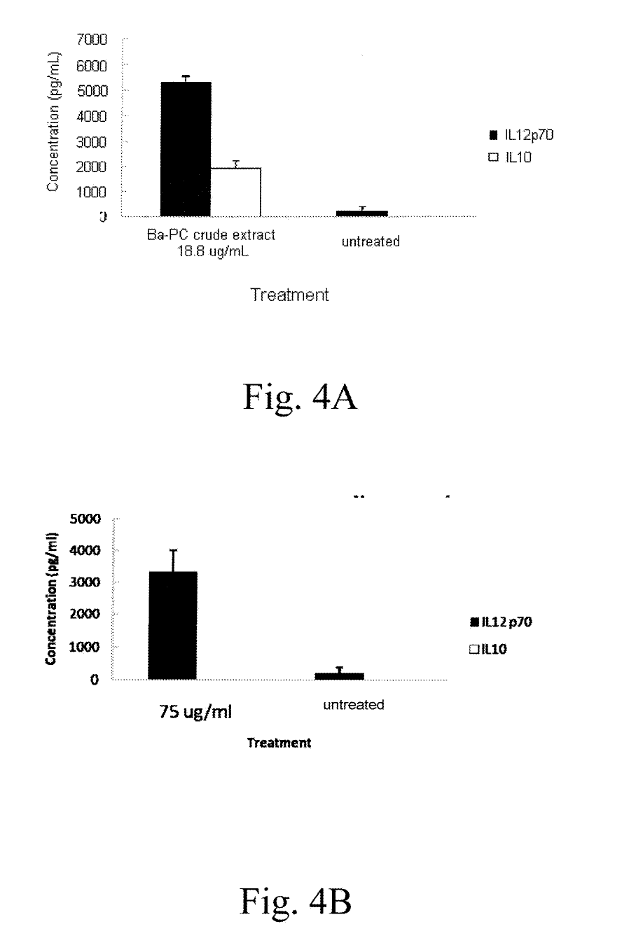

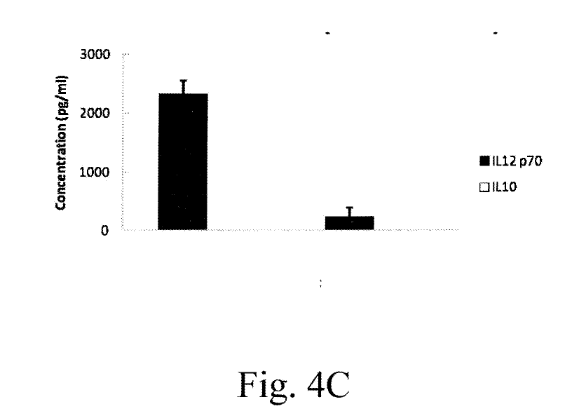

As shown in FIGS. 4A and 4B, Ba-PC crude extract-treated DCs produce considerable amounts of IL-12 p70 (5000 pg/ml), a Th1 cytokine, and IL-10 (2000 pg/ml), a Th2 cytokine Contrarily, as shown in FIG. 1C, IL-10 production by endotoxin-free Ba-PC crude extract treated DCs was not observed. The results indicated that Ba-PC crude extract was capable of modulating BMDCs to elicit a Th1 immune responses as a purified Ba-PC.

### Example 2

### Analysis of Cell Marker Expression and Cytokine Production of Ba-PC-Pulsed Bone Marrow Derived Dendritic Cells

LPS, PS-G or Ba-PC-treated and untreated bone marrow derived dendritic cells (BMDCs) were prepared and cultured as described in “3. Isolation and modulation of mouse dendritic cells from bone marrow cultures”. The cultured conditional medium obtained at 24 hrs and 48hrs after initial culturing was collected and analyzed by ELISA. Expression of surface markers (CD11c, CD80, CD86, IA^{d}, CD40 and CD205) of DCs was analyzed by flow cytometry as described in “4. Flow cytometry”. DCs cultured with LPS or PS-G served as positive controls.

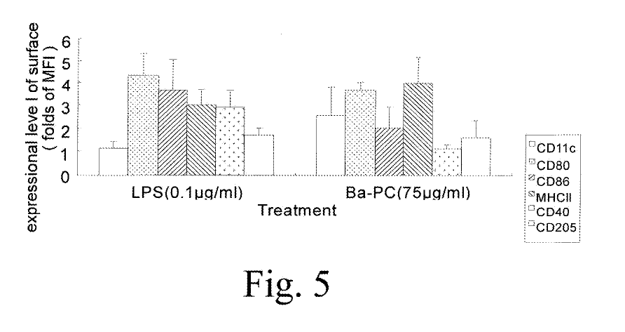

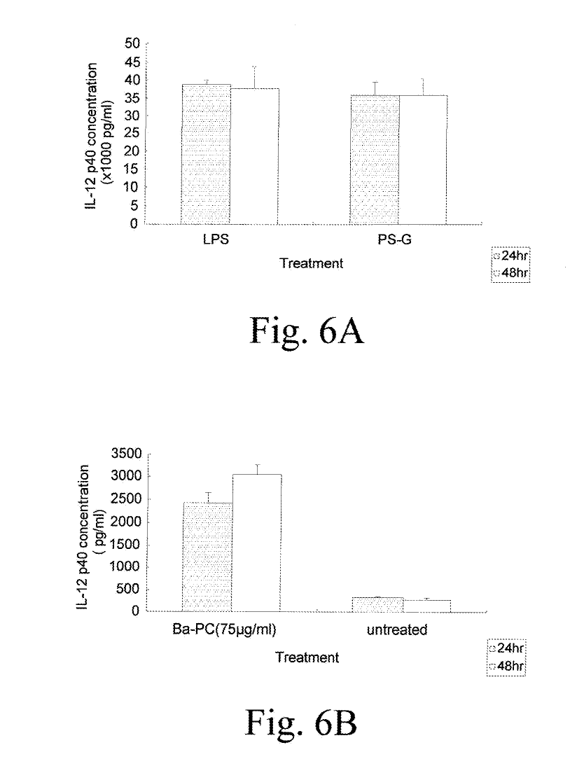

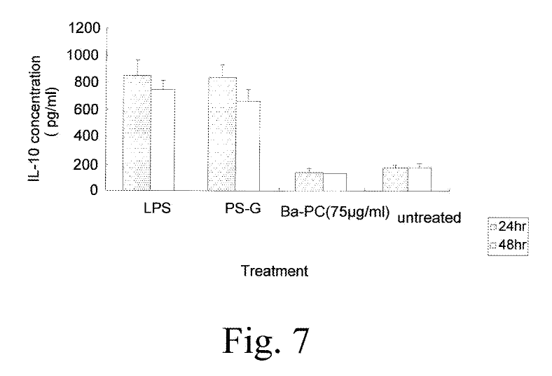

Compared to LPS-treated DCs, as shown in FIG. 5, expression of cell surface markers related to activation and maturation showed Ba-PC-treated DCs increased. Such results suggested Ba-PC had great potential in modulating immune response. Compared to LPS- or PS-G-treated DCs, as shown in FIGS. 6A and 6B, Ba-PC-treated DCs produced considerable amounts of IL-12, a Th1 cytokine Compared to LPS- or PS-G-treated DCs, as shown in FIG. 7, Ba-PC-treated DCs showed almost no production of IL-10, a representative Th2 cytokine This indicated that Ba-PC had great potential in modulating immune response, quite likely switching immune response toward Th1 response.

### Example 3

### Evaluation of Effects of Ba-PC on DC Maturation by Monitoring Endocytosis of DCs

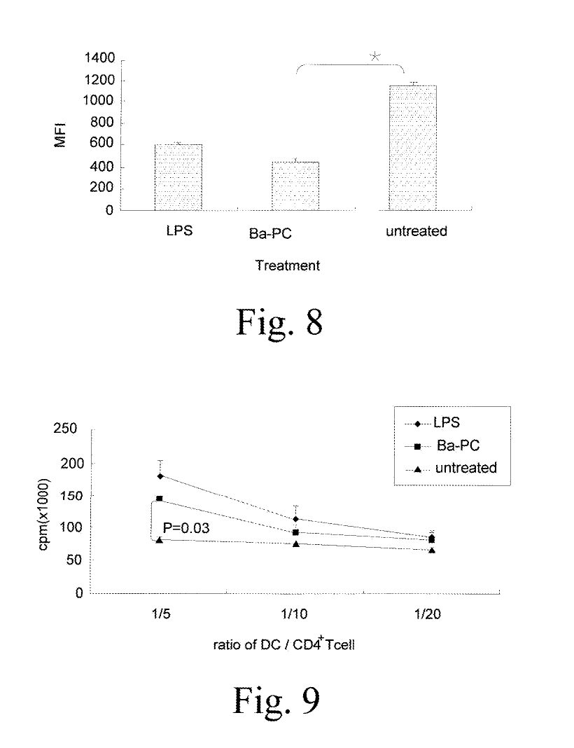

Dextran uptake of Ba-PC treated DCs was monitored as described in “6. Analysis of DC endocytosis” for evaluating capacity of endocytosis. LPS-treated DCs were used as positive control. Untreated DCs were used as negative control. Compared to untreated DCs, as shown in FIG. 8, a capacity of endocytosis of Ba-PC-treated DC was reduced, indicating that a degree of maturity of the DCs was enhanced by Ba-PC treatment.

### Example 4

### Determination of Effects of Ba-PC on CD4⁺ T Cell Proliferation and Cytokine Production

To determine the effects of Ba-PC-treated DCs on activation of T cells, Ba-PC-treated DCs were subject to co-culture with CD4⁺ T cells and proliferation and cytokine production of CD4⁺ T cells were examined as described in “7. Allogenic mixed lymphocyte reactions and cytokine production analysis”. The cytokine production of the CD4⁺ T cells was accessed by the method described in “8. Intracellular cytokine staining” and the cytokine level was measured by ELISA the method described in “5. determination of cytokine expression”. LPS-treated DCs were used as positive control and untreated DCs were used as negative control.

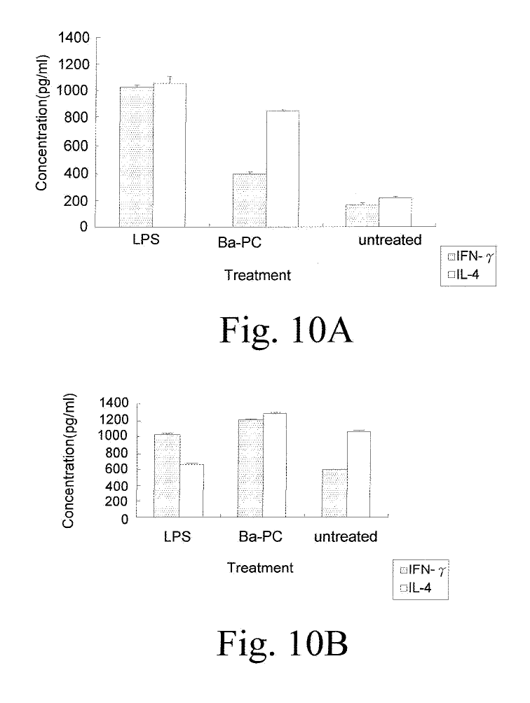

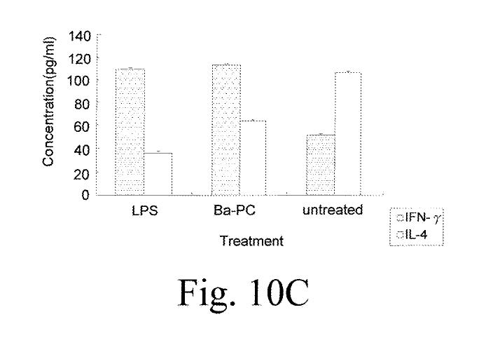

Compared to untreated DCs, as shown in FIG. 9, CD4⁺ T cells co-cultured with Ba-PC-treated DCs showed enhanced proliferation, especially in a ratio of DC/CD4⁺ T cells of 1/5 to 1/10, indicating that Ba-PC can promote activation of CD4⁺ T cells. As shown in FIGS. 10A to 10C, Ba-PC-treated DCs can largely induce CD4⁺ T cells to produce IFN-γ. Furthermore, production of IFN-γ of CD4⁺ T cells increased with the time (48 hours, FIG. 10A; 72 hours, FIG. 10B; and 96 hours, FIG. 10C) of co-culturing of Ba-PC-treated DCs and CD4⁺ T cells, which was consistent with previous observation showed in FIG. 6B that Ba-PC-treated DCs can effectively stimulate Th1 response.

### Example 5

### Determining Type of Immune Response of Mice Treated with OVA in Combination with Ba-PC

To determine whether Ba-PC can effectively modulate immune response in an animal model, mice treated with OVA and Ba-PC (denoted as Ba-PC combination-treated group) were prepared and analyzed as described in “9. Induction of allergic airway inflammation with OVA and analysis of airway hyperresponsiveness (AHR)”. Sera IgG1, IgG2a and IgE were examined as described in “10. Detection and determination of OVA-specific antibody”. Mice treated with OVA in combination with PS-G (denoted as PS-G combination-treated group) were used as a positive control. Mice treated with OVA alone (denoted as OVA alone group) were used as disease control. Mice administered with PBS alone were used as normal control.

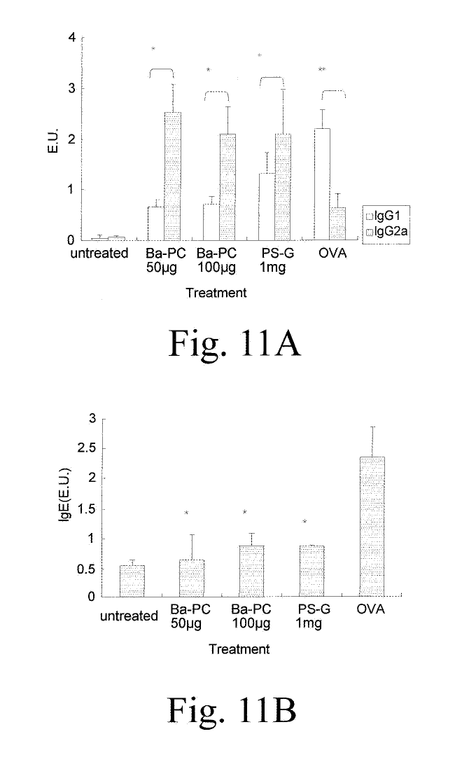

As shown in FIGS. 11A and 11B, in mice treated with OVA in combination with Ba-PC, higher amounts of IgG2a and lower amounts of IgG1 or IgE were observed, which was consistent with previous speculation that Ba-PC-treated DCs can effectively stimulate Th1 response.

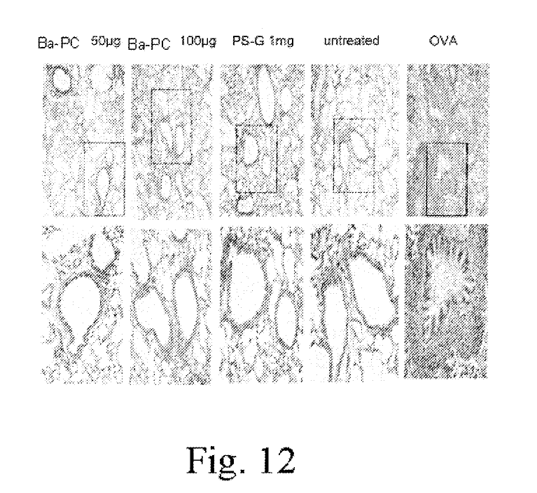

As shown in FIG. 12, compared to OVA-immunized alone mice, mice treated with OVA in combination with Ba-PC exhibited mild bronchial inflammation, fewer occurrences of infiltrated inflammatory cells and unnoticeable thickening of bronchial epithelial cells, indicating that Ba-PC contributed to alleviate severity of bronchial inflammation.

### Example 6

### Examination of Cellular Composition in the BAL Fluid from Mice Treated with OVA in Combination with Ba-PC

To further evaluate whether Ba-PC can modulate recruitment of inflammatory cells in the airway, different cells in BAL fluid obtained from mice treated with OVA in combination with Ba-PC were accessed as described in “12. Preparation and analysis of cellular composition and cytokine level of BAL fluid” and percentage of eosinophils in all cells in BALF was calculated. Mice treated with OVA in combination with PS-G were used as positive control. Mice treated with OVA alone were used as disease control. Mice administered with PBS alone were used as normal control.

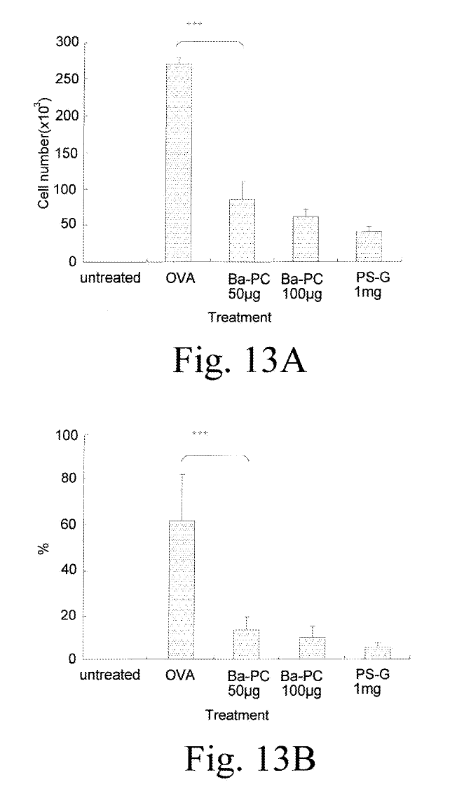

As shown in FIG. 13A and 13B, infiltration of eosinophils in the airway were downregulated in mice treated with OVA in combination with Ba-PC or PSG A statistically significant difference was shown between Ba-PC- or PS-G- and combination-treated group OVA alone group (***: p<0.0001). In contrast, mice treated with OVA alone showed a significant infiltration of eosinophils. Such results agree with previous conclusion that Ba-PC can alleviate severity of bronchial inflammation.

### Example 7

### Examination of Airway Hyperresponsiveness in Mice Treated with OVA in Combination with Ba-PC

To further explore whether Ba-PC can protect against airway hyperactivity in murine models of allergic airway disease, mice treated with OVA in combination with Ba-PC were prepared and analyzed for airway hyperresponsiveness (AHR) by comparison to the OVA-immunized mice without any treatment as described in “9. Induction of allergic airway inflammation with OVA and analysis of airway hyperresponsiveness (AHR)” and “11. Determination of airway function”. Mice treated with OVA in combination with PS-G were used as positive control. Mice treated with OVA alone were used as disease control. Mice administered with PBS alone were used as normal control.

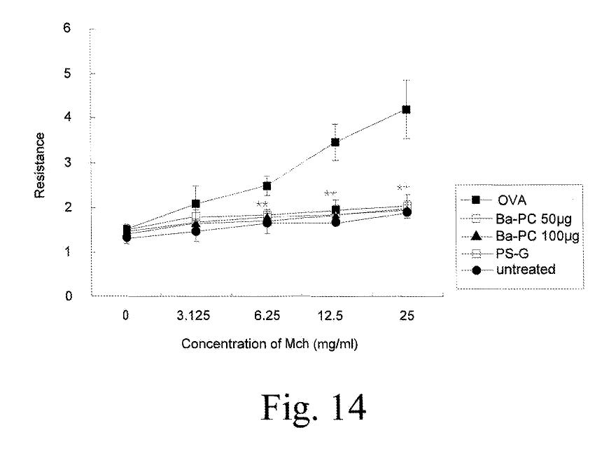

As shown in FIG. 14, the mice treated with OVA alone showed severe airway hyperresponsiveness, while mice treated with OVA in combination with Ba-PC had a significant decrease in airway hyperresponsiveness, as accessed by their response to increasing doses of inhaled methacholine (Mch). The mice treated with OVA in combination with PSG also had decreased airway hyperresponsiveness. Such results indicate that Ba-PC could alleviate the severity of bronchial inflammation.

### Example 8

### Examination of Cytokine Kevels in Brochoalveolar Lavage Fluid from Mice Treated with OVA in Combination with Ba-PC

The IL-4, IL-10, IL-5, eotaxin and IL-13 expression in the BAL fluid derived from each group of mice with different treatment was measured as described in “12. Preparation and analysis of cellular composition and cytokine level of BAL fluid”. OVA-immunized mice treated with PSG served as positive control. Mice treated with OVA alone were used as disease control. Mice administered with PBS alone were used as normal control.

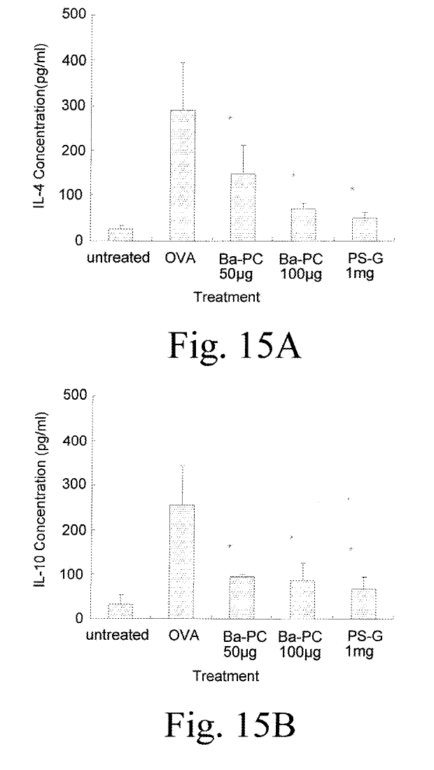

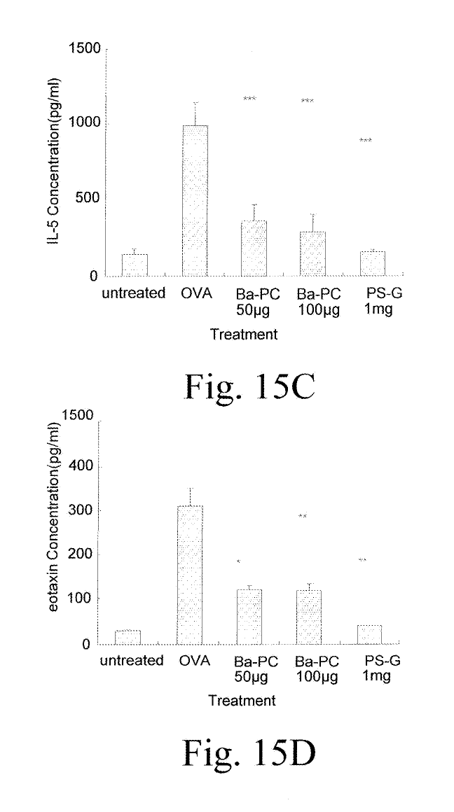

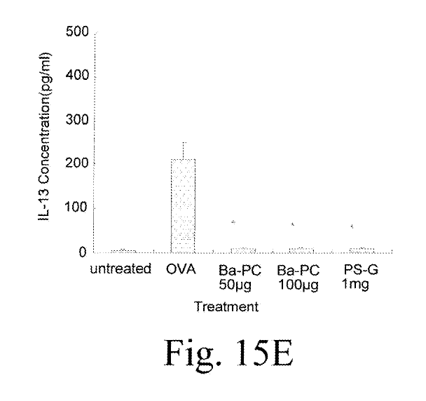

As shown in FIG. 15A to 15E, compared to OVA-immunized mice, levels of IL-4, IL-10, IL-5, eotaxin and IL-13 tended to decrease in groups of OVA in combination with BaPC-immunized mice. Such results indicated that Ba-PC could affect immune response through alteration of cytokine production of BAL fluid and Ba-PC could alleviate severity of bronchial inflammation.

### Example 9

### Examination of Cytokine Levels in the Splenocytes from Mice Treated with OVA in Combination with Ba-PC

To examine effects of Ba-PC on modulating antigen-specific immune response in OVA-immunized mice, splenocytes obtained from mice treated with OVA in presence or absence of Ba-PC as described previously were co-cultured with OVA to induce OVA-specific T cell activation. Culture supernatant was collected at 5 days after stimulation and was analyzed for IL-4, IL-5, IL-10, IL-13 and IFN-γ by ELISA as described in “12. Preparation and analysis of cellular composition and cytokine level of BAL fluid”.

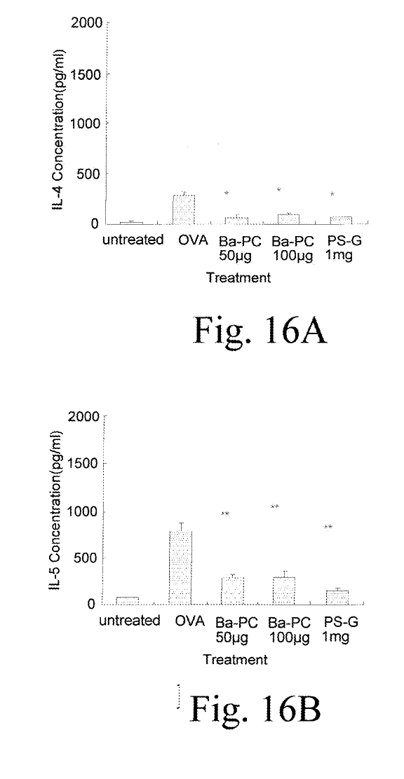

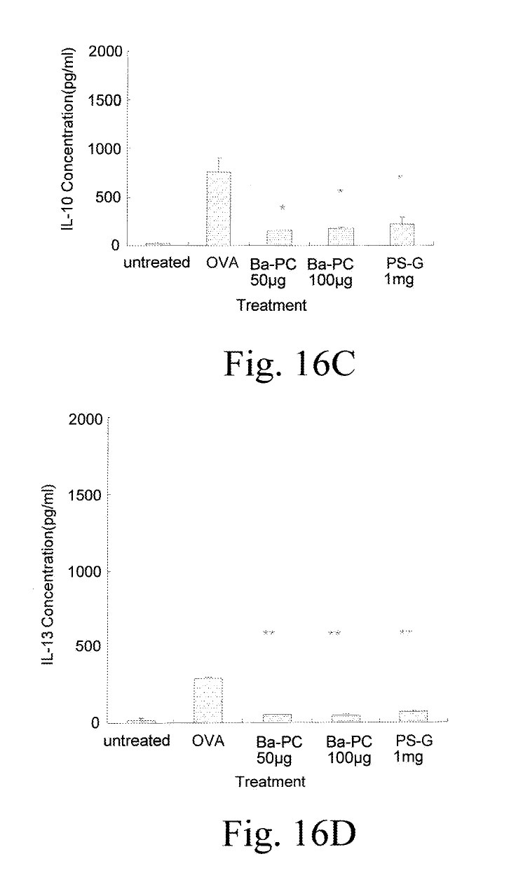

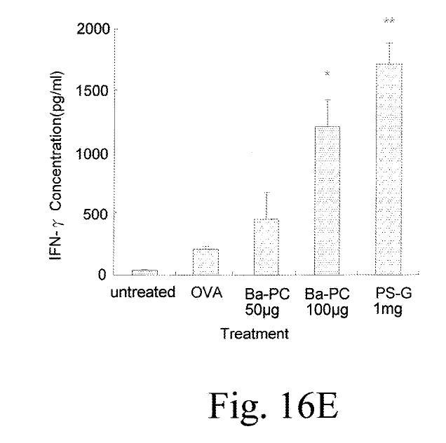

As shown in FIG. 16A to 16E, cytokine production levels of IL-4, IL-5, IL-10 and IL-13 from antigen-specific T cells tended to decrease in the group of OVA in combination with Ba-PC-immunized mice, while cytokine production level of IFN-γ tended to increase in the same of group, suggesting that Ba-PC mounts a Th1-type immune response by promoting a Th1-type milieu of cytokine production. Such results of in vivo experiments demonstrated that Ba-PC could effectively decrease Th2-type cytokine production and alleviate Th2-type immune response.

### Example 10

### Examination of Proliferative Activity of OVA-Specific T Cells from Mice Treated with OVA in Combination with Ba-PC

CD4⁺ T cells obtained from mice treated with OVA with or without Ba-PC were stimulated with 5 μg/ml of OVA in vitro and their proliferation was analyzed by ³H-thymidine incorporation analysis as described in “7. Allogenic mixed lymphocyte reactions and cytokine production analysis”. Mice treated with OVA in combination with PS-G served as a positive control.

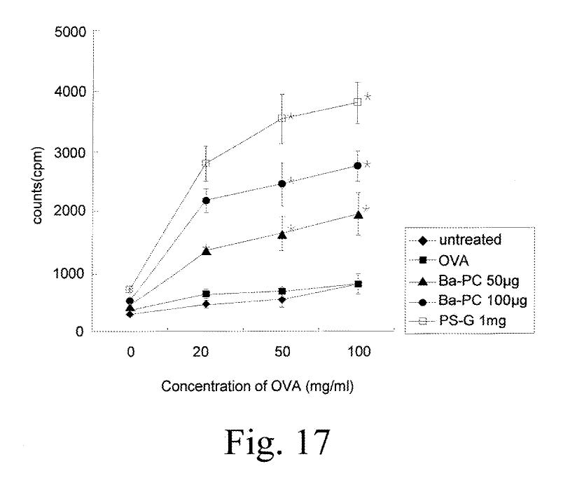

As shown in FIG. 17, proliferative activity of OVA-specific CD4⁺ T cells from mice treated with OVA in combination with Ba-PC was higher than that of OVA-specific CD4⁺ T cells derived from OVA-immunized mice without any treatment. Such result indicated that Ba-PC enhanced the proliferative activity of antigen-specific T cells in mice.

All patents, patent applications, and literature cited in the specification were incorporated by reference in their entirety. In the case of any inconsistencies, the present disclosure, including any definitions therein will prevail.

Although preferred embodiments of the invention have been described in detail, certain variations and modifications is apparent to those skilled in the art, including embodiments that do not provide all of the features and benefits described herein. Accordingly, the scope of the present invention is not to be limited by the illustrations or the foregoing descriptions thereof, but rather solely by reference to the appended claims.

What is claimed is:

1. A method for treating or alleviating allergic disease in a mammal in need thereof, comprising:
administering to the mammal a therapeutically effective amount of a pharmaceutical composition comprising phycocyanin.

2. The method according to claim 1, wherein the allergic disease is selected from the group consisting of: asthma, allergic rhinitis, eczema, psoriasis, atopic dermatitis, rheumatoid arthritis, inflammatory bowel disease, Crohn's disease, ulcerative colitis, chronic obstructive pulmonary disease, conjunctivitis, nasal congestion and urticaria.

3. The method according to claim 2, wherein the therapeutically effective amount is between 0.1 mg per kg per day and 50 mg per kg per day.

4. The method according to claim 1, wherein the phycocyanin is obtained from alga selected from the group consisting of algae belonging to genus of Porphyra and Bangia and combination thereof.

5. The method according to claim 1, wherein the phycocyanin is obtained from Bangia atropupurea.

6. The method according to claim 1, wherein the allergic disease is allergic airway disease.

7. The method according to claim 6, wherein the phycocyanin is obtained from Bangia atropupurea.

8. The method according to claim 7, wherein the therapeutically effective amount is between 0.1 mg per kg per day and 50 mg per kg per day.

9. The method according to claim 7, wherein the pharmaceutical composition is administered orally, buccally, topically, inhalationally or intranasally to the mammal.

10. The method according to claim 7, wherein the pharmaceutical composition is administered intravenously, subcutaneously, or intramuscularly to the mammal.

11. A method for modulating balance between Th1 and Th2 immune response in a mammal in need thereof, comprising:
administering to the mammal an effective amount of phycocyanin,
wherein immune response of the mammal is skewed toward the Th1 immune response.

12. The method according to claim 11, wherein the mammal is suffering from allergic disease.

13. The method according to claim 11, wherein the mammal is suffering from allergic airway disease.

14. The method according to claim 13, wherein the allergic airway disease is selected from the group consisting of: asthma, allergic rhinitis and allergic pneumonia.

15. The method according to claim 11, wherein the phycocyanin is obtained from alga selected from the group consisting of algae belonging to Porphyra, Bangia and combination thereof.

16. The method according to claim 11, wherein the phycocyanin is obtained from Bangia atropupurea.

17. The method according to claim 16, wherein the effective amount is between 0.1 mg per kg per day and 50 mg per kg per day.

18. A pharmaceutical composition for alleviating allergic disease, comprising a therapeutically effective amount of phycocyanin and a pharmaceutically acceptable carrier or excipient therefor.

19. The pharmaceutical composition according to claim 18, wherein the therapeutically effective amount is between 0.1 mg per kg and 50 mg per kg.

20. The pharmaceutical composition according to claim 18, wherein the pharmaceutical composition is formulated for administration by oral, topical, parenteral, intramuscular, intranasal, subcutaneous or intravenous routes.
